(19) United States
(12) Patent Application Publication
(10) Pub. No.: US 2018/0338693 A1
(43) Pub. Date: Nov. 29, 2018

(54) USE OF A MULTI-COMPARTMENT BLADDER IN A FINGER CUFF

(71) Applicant: Edwards Lifesciences Corporation, Irvine, CA (US)

(72) Inventors: Peiyuan Li, Amsterdam (NL); Olaf Schraa, Amsterdam (NL); Jacobus Jozef Gerardus Maria Settels, De Hoef (NL); Geertruida Lucretia van der Sar, Alphen aan den Rijn (NL)

(21) Appl. No.: 15/985,033

(22) Filed: May 21, 2018

Related U.S. Application Data
(60) Provisional application No. 62/510,557, filed on May 24, 2017.

(51) Int. Cl. A61B 5/022; A61B 5/0225

(52) U.S. Cl. CPC A61B 5/02241; A61B 5/02141; A61B 5/02255

(57) ABSTRACT

Disclosed is a finger cuff apparatus connectable to a patient's finger to be used in blood pressure measurement using volume clamping. The finger cuff apparatus may comprise a first bladder and a second bladder or multiple bladders.

### CROSS-REFERENCE TO RELATED APPLICATIONS

This application claims priority to U.S. Provisional Patent Application No. 62/510,557, filed May 24, 2017, the entire contents of which are incorporated herein by reference.

### BACKGROUND

### Field

Embodiments of the invention relate to a finger cuff used for non-invasive blood pressure measurement.

### Relevant Background

Volume clamping is a technique for non-invasively measuring blood pressure in which pressure is applied to a subject's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure.

This may be accomplished by a finger cuff that is arranged around a finger of a patient. The finger cuff may include an infrared light source, an infrared sensor, and an inflatable bladder. With a finger cuff, by inflating the bladder in the finger cuff, a pressure is exerted on the outside of a finger artery. If the bladder pressure is higher than the pressure inside the artery, it will compress the artery and the amount of light registered by the infrared sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure.

In many current implementations, the finger cuff is a stiff, conically shaped apparatus that is still flexible enough to be wrapped around the finger. Due to differences in finger shapes (e.g., the finger can be straight, have more pronounced knuckles, may change with age, etc.), the pressure of the finger cuff may not be evenly distributed over the finger, which can result in patient discomfort, especially at the edge of the finger cuff and/or around the knuckles, where the bone is close to the skin surface.

Other than patient discomfort, an ill-fitted finger cuff may decrease measurement accuracy as more accurate measurements are obtained when the bladder in the finger cuff is in more close contact with the skin such that pressure is more evenly distributed over the finger. For example, measurements on a finger with relatively big knuckles may be less accurate than measurements on a finger with less prominent knuckles.

Additionally, the pressure in a large finger cuff is difficult to control due to the large bladder volume. Measurement accuracy may be compromised when the bladder volume is too large.

Moreover, the measurement accuracy of existing finger cuffs is susceptible to application errors. For example, when the finger cuff is wrapped too loosely or too tightly around the finger, the measurements may be less accurate. Also, in another example, when a conically shaped finger cuff is applied in the wrong orientation (e.g., upside down), the measurements may also be less accurate.

### SUMMARY

Embodiments of the invention may relate to a finger cuff apparatus connectable to a patient's finger to be used in blood pressure measurement using volume clamping. The finger cuff apparatus may comprise: a first bladder; and a second bladder or multiple bladders.

### BRIEF DESCRIPTION OF THE DRAWINGS

FIG. 1 is a diagram of an example of a blood pressure measurement system.

FIGS. 2A and 2B are cross-sectional views of a conventional, single bladder finger cuff wrapped around a finger.

FIGS. 3A and 3B are cross-sectional views of a finger cuff wrapped around a finger according to one embodiment of the invention.

FIGS. 4A and 4B are cross-sectional views of a finger cuff wrapped around a finger according to one embodiment of the invention.

FIGS. 5A and 5B are cross-sectional views of a finger cuff wrapped around a finger according to one embodiment of the invention.

### DETAILED DESCRIPTION

Embodiments of the invention may relate to a finger cuff apparatus connectable to a patient's finger to be used in blood pressure measurement using volume clamping. The finger cuff apparatus may comprise: a first bladder; and a second bladder or multiple bladders. The first bladder is inflatable to be used in the blood pressure measurement using volume clamping. Further, as will be described, the second bladder or multiple bladders may be fixed sized bladders or may be adjustable sized bladders or inflatable bladders. As will be described, one or more fixed sized bladders that may or may not be pneumatically or hydraulically connected may be arranged in close proximity to an inflatable bladder or another fixed sized bladder.

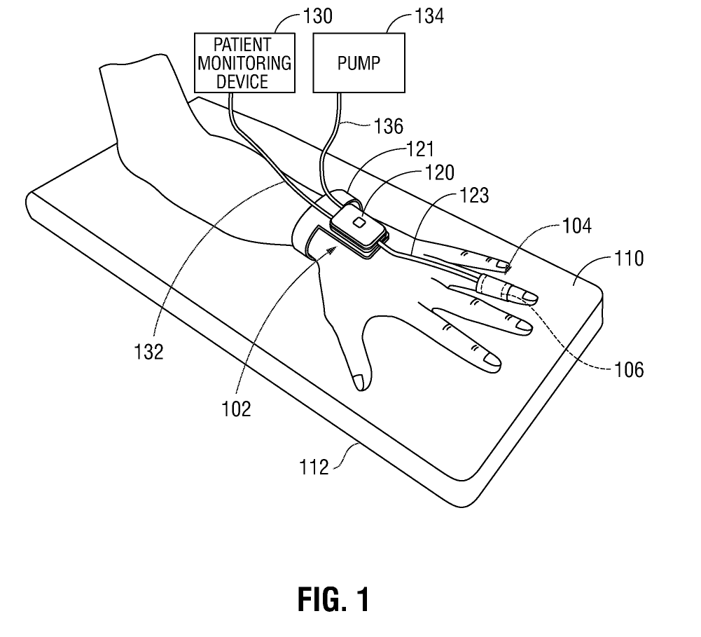

With reference to FIG. 1, an example of an environment in which a finger cuff 104 may be implemented will be described. As an example, a blood pressure measurement system 102 that includes a finger cuff 104 that may be attached to a patient's finger and a blood pressure measurement controller 120 that may be attached to the patient's body (e.g., a patient's wrist or hand) is shown. The blood pressure measurement system 102 may further be connected to a patient monitoring device 130, and, in some embodiments, a pump 134. Further, finger cuff 104 may include a bladder 106 and an LED-PD pair (not shown).

In one embodiment, the blood pressure measurement system 102 may include a pressure measurement controller 120 that includes: a small internal pump, a small internal valve, a pressure sensor, and control circuity. In this embodiment, the control circuitry may be configured to: control the pneumatic pressure applied by the internal pump to the bladder of the finger cuff 104 to replicate the patient's blood pressure based upon measuring the pleth signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure sensor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder 106 of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the pleth signal received from the LED-PD pair of the finger cuff 104 and measuring the patient's blood pressure by monitoring the pressure of the bladder may be controlled by the blood pressure measurement controller 120 and/or a remote computing device and/or the pump 134 and/or the patient monitoring device 130. In some embodiments, a blood pressure measurement controller 120 is not used at all and there is simply a connection from the tube 123 to the finger cuff 104 from a remote pump 134 having a remote pressure regulatory system, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder 106 of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). Any kind of pressure generating and regulating system that can be used, including but not limited to the blood pressure measurement controller, may be described simply as a pressure generating and regulating system. As a further example, in some embodiments, there may be no blood pressure measurement controller, at all, and a remote pump 134 that is controlled remotely may be directly connected via a tube to finger cuff 104 to provide pneumatic pressure to the finger cuff 104.

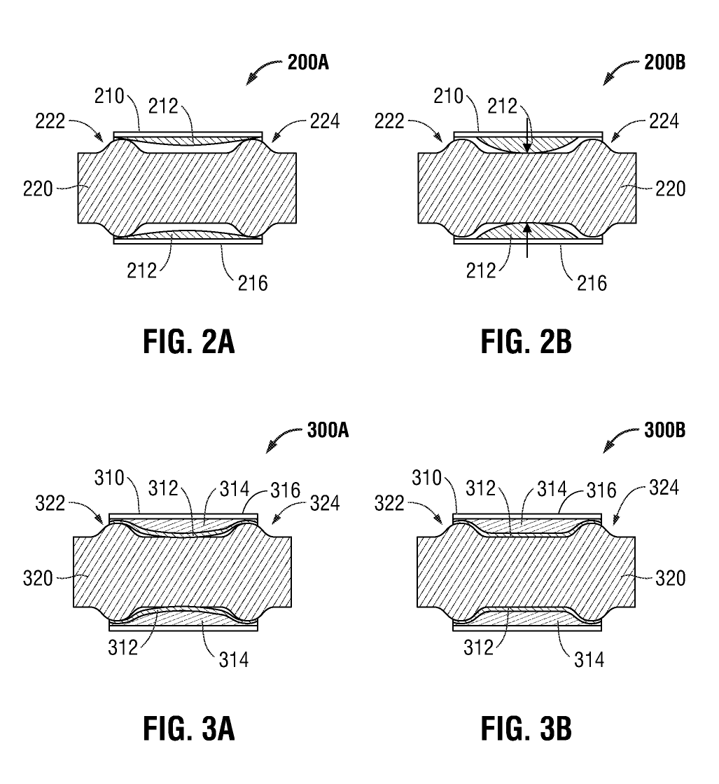

Referring to FIGS. 2A and 2B, cross-sectional views 200A, 200B of a conventional single bladder finger cuff wrapped around a finger are shown. FIG. 2A shows a finger cuff 210 wrapped around a finger 220 when a single bladder 212 is not inflated (e.g., when a blood pressure measurement is not in progress), while FIG. 2B shows the finger cuff 210 wrapped around the finger 220 when the bladder 212 is inflated (e.g., when a blood pressure measurement is progress). The bladder 212 may be arranged within the finger cuff 210 so that the bladder 212 approximately wraps around the finger 220 when the finger cuff 210 is wrapped around the finger 220. Two knuckles 222, 224 of the finger 220 come into contact with the finger cuff 210 at the edges of the finger cuff 210. As can be seen in FIGS. 2A and 2B, patient discomfort may be experienced around where the knuckles 222, 224 come into contact with the finger cuff 210. Moreover, the finger cuff pressure may not be evenly distributed over the finger 220 when the bladder 212 is inflated.

Referring to FIGS. 3A and 3B, cross-sectional views 300A, 300B of a finger cuff wrapped around a finger according to one embodiment of the invention are shown. FIG. 3A shows a finger cuff 310 wrapped around a finger 320 when a blood pressure measurement is not in progress, while FIG. 3B shows the finger cuff 310 wrapped around the finger 320 when a blood pressure measurement is progress. The finger cuff 310 comprises two bladders—a first bladder 312 and a second bladder 314, which are arranged next to each other in parallel. The first bladder 312 is closer to the finger 320 and the second bladder 314 is outside the first bladder 312 when the finger cuff 310 is wrapped around the finger 320. As can be seen in FIG. 3B, the first bladder 312 may be inflated for volume clamping during blood pressure measurements. It should be appreciated that the second bladder 314 may serve as a cushion between the frame 316 of the finger cuff 310 and the knuckles 322, 324, therefore improving patient comfort as well as helping evenly distribute the finger cuff pressure over the finger 320 during blood pressure measurements. Thus, in this embodiment, the second bladder 314 is a fixed sized bladder and has a fixed volume. It should be appreciated that multiple fixed sized bladders having fixed volumes may be used.

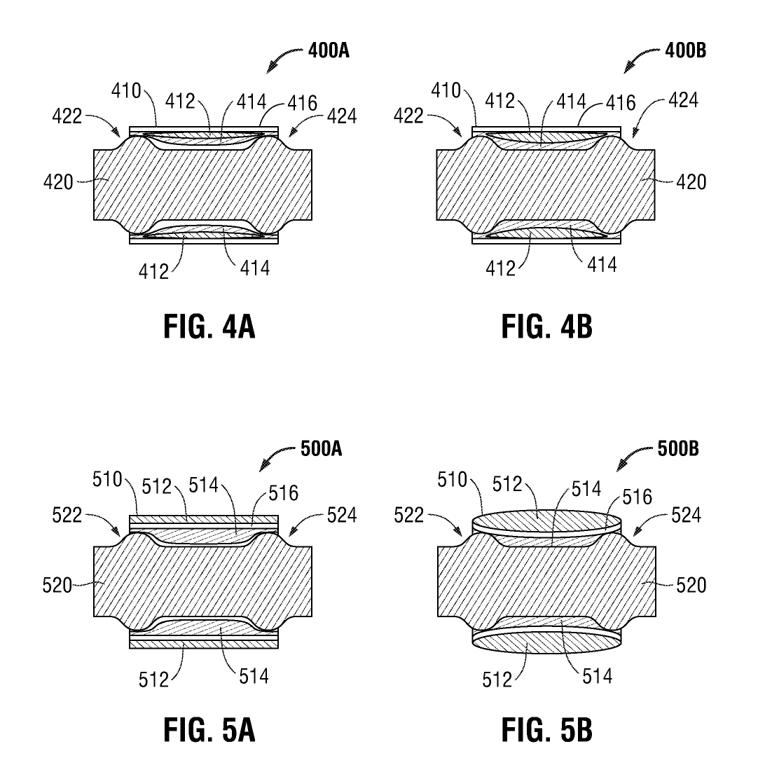

Referring to FIGS. 4A and 4B, cross-sectional views 400A, 400B of a finger cuff wrapped around a finger according to one embodiment of the invention are shown. FIG. 4A shows a finger cuff 410 wrapped around a finger 420 when a blood pressure measurement is not in progress, while FIG. 4B shows the finger cuff 410 wrapped around the finger 420 when a blood pressure measurement is progress. The finger cuff 410 comprises two bladders—a first bladder 412 and a second bladder 414. The first bladder 412 may be deposited fully within the second bladder 414. Therefore, the second bladder 414 is closer to the finger 420 than the first bladder 412. As can be seen in FIG. 4B, the first bladder 412 may be inflated during blood pressure measurements. As the first bladder 412 expands, it further deforms the second bladder 414 and pushes the second bladder 414 toward the finger 420, thereby creating the finger cuff pressure over the finger 420 that performs volume clamping. It should be appreciated that the second bladder 414 may serve as a cushion between the frame 416 of the finger cuff 410 and the knuckles 422, 424, therefore improving patient comfort as well as helping evenly distribute the finger cuff pressure over the finger 420 during blood pressure measurements. Thus, in this embodiment, the second bladder 414 is a fixed sized bladder and has a fixed volume. It should be appreciated that multiple fixed sized bladders having fixed volumes may be used.

Referring to FIGS. 5A and 5B, cross-sectional views 500A, 500B of a finger cuff wrapped around a finger according to one embodiment of the invention are shown. FIG. 5A shows a finger cuff 510 wrapped around a finger 520 when a blood pressure measurement is not in progress, while FIG. 5B shows the finger cuff 510 wrapped around the finger 520 when a blood pressure measurement is progress. The finger cuff 510 comprises two bladders—a first bladder 512 and a second bladder 514. The first bladder 512 and the second bladder 514 may be arranged in parallel on opposite sides of the frame 516 of the finger cuff 510: the first bladder 512 may be arranged outside the frame 516 of the finger cuff 510, and the second bladder 514 may be arranged inside the frame 516 of the finger cuff 510. Therefore, the second bladder 514 is closer to the finger 520. As can be seen in FIG. 5B, the first bladder 512 may be inflated during blood pressure measurements. As the first bladder 512 expands, it deforms the frame 516 and the second bladder 514 of the finger cuff 510 and pushes the frame 516 and the second bladder 514 toward the finger 520, thereby creating the finger cuff pressure over the finger 520 that performs volume clamping. It should be appreciated that the second bladder 514 may serve as a cushion between the frame 516 of the finger cuff 510 and the knuckles 522, 524, therefore improving patient comfort as well as helping evenly distribute the finger cuff pressure over the finger 520 during blood pressure measurements. As been described, the second bladder 514 is a fixed sized bladder and has a fixed volume. It should be appreciated that multiple fixed sized bladders having fixed volumes may be used.

Therefore, as has been described, as one example, a finger cuff may comprise a first bladder and a second bladder, and during blood pressure measurements, the first bladder is inflated to effect the finger cuff pressure over the finger such that volume clamping is performed. The second bladder may serve as a cushion between the frame of the finger cuff and the finger. In some embodiments, the second bladder may be a fixed sized bladder and have fixed volume. As has been described, multiple bladders being of fixed sized and having fixed volume may be utilized. In other embodiments, the second bladder or multiple bladders may be used that have an adjustable volume and that may be adjustable in size to perform better fitting and/or to be used in volume clamping (similar to the first bladder). The first and second or multiple bladders may be arranged next to each other on the inner side of the frame of the finger cuff, or may be arranged on opposite sides of the frame of the finger cuff. The first bladder and/or the second bladder and/or multiple bladders may be filled/fillable with either air or a liquid and may be fixed sized/volume implementations or may be adjustable in size/volume implementations. Also, it should be appreciated that any number of inflatable and/or fixed sized bladders may be utilized dependent upon design considerations. Additionally, it should be appreciated that in some embodiments the first bladder, the second bladder, and/or multiple bladders may be arranged in the form of a single bladder with multiple layers and multiple compartments.

Therefore, embodiments of the invention are related to an improved finger cuff comprising a plurality of bladders. With the plurality of bladders, patient comfort is improved due to a better fit of the cuff with the finger shape, especially around the knuckles. Measurement accuracy may be improved due to a better and faster pressure transfer from the bladder to the finger. When the total volume of the bladders is partially filled with an incompressible medium (e.g., a fluid such as water or gel), the volume in the inflatable part of the bladder can be smaller, and the pressure in the finger cuff can be more easily controlled, delivering an even higher measurement accuracy. Furthermore, with the plurality of bladders, measurement accuracy is less susceptible to application errors because at least one bladder is used to fill up the gap between the finger and the finger cuff prior to the measurement. Moreover, a finger cuff with a plurality of bladders as described above does not have to be conically shaped, thus it can be worn in either orientation. The tightness of the finger cuff also becomes less critical.

It should be appreciated that aspects of the invention previously described may be implemented in conjunction with the execution of instructions by processors, circuitry, controllers, control circuitry, etc. As an example, control circuity may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A finger cuff apparatus connectable to a patient's finger to be used in blood pressure measurement using volume clamping, comprising:
a first bladder; and
a second bladder or multiple bladders.

2. The finger cuff apparatus of claim 1, wherein the first bladder is inflatable to be used in the blood pressure measurement using volume clamping.

3. The finger cuff apparatus of claim 1, wherein the second bladder or multiple bladders have an adjustable volume.

4. The finger cuff apparatus of claim 1, wherein the second bladder or multiple bladders have a fixed volume.

5. The finger cuff apparatus of claim 4, wherein the second bladder or multiple bladders are filled with a liquid.

6. The finger cuff apparatus of claim 1, wherein the second bladder or multiple bladders serve as a cushion between a frame of the finger cuff apparatus and a knuckle of the patient's finger.

7. The finger cuff apparatus of claim 1, wherein the first and second or multiple bladders are arranged next to each other on an inner side of a frame of the finger cuff apparatus.

8. The finger cuff apparatus of claim 1, wherein the first bladder is arranged inside the second or multiple bladders.

9. The finger cuff apparatus of claim 1, wherein the first and second or multiple bladders are arranged on opposite sides of a frame of the finger cuff apparatus.

10. The finger cuff apparatus of claim 9, wherein the first bladder is arranged on an outer side of the frame of the finger cuff apparatus, and the second or multiple bladders are arranged on an inner side of the frame of the finger cuff apparatus.

11. The finger cuff apparatus of claim 1, wherein the first and second or multiple bladders are arranged in the form of a single bladder with multiple layers and multiple compartments.

12. A blood pressure measurement system to measure a patient's blood pressure comprising:
a pump; and
a finger cuff apparatus connectable to the pump and to a patient's finger to be used in the blood pressure measurement using volume clamping, including: a first bladder and a second bladder or multiple bladders.

13. The blood pressure measurement system of claim 12, wherein the first bladder is inflatable to be used in the blood pressure measurement using volume clamping.

14. The blood pressure measurement system of claim 12, wherein the second bladder or multiple bladders have an adjustable volume.

15. The blood pressure measurement system of claim 12, wherein the second bladder or multiple bladders have a fixed volume.

16. The blood pressure measurement system of claim 15, wherein the second bladder or multiple bladders are filled with a liquid.

17. The blood pressure measurement system of claim 12, wherein the second bladder or multiple bladders serve as a cushion between a frame of the finger cuff apparatus and a knuckle of the patient's finger.

18. The blood pressure measurement system of claim 12, wherein the first and second or multiple bladders are arranged next to each other on an inner side of a frame of the finger cuff apparatus.

19. The blood pressure measurement system of claim 12, wherein the first bladder is arranged inside the second or multiple bladders.

20. The blood pressure measurement system of claim 12, wherein the first and second or multiple bladders are arranged on opposite sides of a frame of the finger cuff apparatus.

21. The blood pressure measurement system of claim 20, wherein the first bladder is arranged on an outer side of the frame of the finger cuff apparatus, and the second or multiple bladders are arranged on an inner side of the frame of the finger cuff apparatus.

22. The blood pressure measurement system of claim 12, wherein the first and second or multiple bladders are arranged in the form of a single bladder with multiple layers and multiple compartments.

23. A method to measure a patient's blood pressure comprising:
connecting a finger cuff apparatus to a pump; and
connecting the finger cuff apparatus to a patient's finger to be used in the blood pressure measurement, wherein, the finger cuff apparatus includes a first bladder and a second bladder or multiple bladders.

24. The method of claim 23, wherein the first bladder is inflatable to be used in the blood pressure measurement using volume clamping.

25. The method of claim 23, wherein the second bladder or multiple bladders have an adjustable volume.

26. The method of claim 23, wherein the second bladder or multiple bladders have a fixed volume.

27. The method of claim 26, wherein the second bladder or multiple bladders are filled with a liquid.

28. The method of claim 23, wherein the second bladder or multiple bladders serve as a cushion between a frame of the finger cuff apparatus and a knuckle of the patient's finger.

29. The method of claim 23, wherein the first and second or multiple bladders are arranged next to each other on an inner side of a frame of the finger cuff apparatus.

30. The method of claim 23, wherein the first bladder is arranged inside the second or multiple bladders.

31. The method of claim 23, wherein the first and second or multiple bladders are arranged on opposite sides of a frame of the finger cuff apparatus.

32. The method of claim 31, wherein the first bladder is arranged on an outer side of the frame of the finger cuff apparatus, and the second or multiple bladders are arranged on an inner side of the frame of the finger cuff apparatus.

33. The method of claim 23, wherein the first and second or multiple bladders are arranged in the form of a single bladder with multiple layers and multiple compartments.
